(19) United States
(12) Patent Application Publication
(10) Pub. No.: US 2004/0234473 A1
(43) Pub. Date: Nov. 25, 2004

(54) Compositions comprising at least one organic polymer and a mixture of volatile and nonvolatile solvents

(75) Inventors: Veronique Ferrari, Maisons-Alfort (FR); Florence Tournilhac, Paris (FR)

Correspondence Address: FINNEGAN, HENDERSON, FARABOW, GARRETT & DUNNER, LLP, 1300 I STREET, NW, WASHINGTON, DC, 20005, US

(73) Assignee: L'OREAL S.A.

(21) Appl. No.: 10/621,534

(22) Filed: Jul. 17, 2003

Related U.S. Application Data
(63) Continuation of application No. 09/618,031, filed on Jul. 17, 2000, now abandoned.

(30) Foreign Application Priority Data
Jul. 16, 1999 (FR) 99 09265

(51) Int. Cl. A61K 7/06; A61K 7/11

(52) U.S. Cl. 424/70.11

(57) ABSTRACT

Compositions, for example cosmetic compositions, comprising at least one organic polymer, such as a crosslinked organosoluble polymer, and a mixture of at least two solvents, one of which is volatile and incompatible with the polymer, and the other of which is nonvolatile and compatible with the polymer. These compositions make it possible to form on keratin materials a deposit whose volume increases over time, without the intervention of an external stimulus. These compositions can be, for example, mascaras, lipsticks or anti-wrinkle formulas in the form of a cream or a foundation. The cosmetic treatment processes using these compositions.

The present invention relates to a composition, such as a cosmetic composition, for the skin, including the scalp, and/or fort he lips of human beings, comprising at least one organic polymer, such as an organosoluble gel which may be crosslinked, and a mixture of volatile and nonvolatile solvents. The composition of the invention can be used as a make-up composition for-keratin materials such as the skin, the lips and superficial body growths, which gives a volume effect.

The application of known cosmetic compositions to keratin materials can lead to a deposit which cain become thinned out over time, by penetration of all or some of the deposited composition into these materials and/or by evaporation of the volatile constituents initially present in the deposit. These phenomena may then reveal defects on the support such as fine lines, pigmentation defects such as blemishes on the hands and face or the loss of color from the lips, and acne rosacea, which is a particular inconvenience when depositing a foundation on the human face. Specifically, one of the aims of a foundation is to camouflage the imperfections (e.g., blemishes and blackheads) of the skin and to unify its complexion.

Moreover, women are looking more and more to remodel their face and/or body, for example, the lips of the face, in order to increase their volume. Hitherto, increasing the volume of certain parts of the face or the body has been achieved by injecting substances such as silicone gels. This type of remodeling generally takes place under local or even general anesthesia, which may disrupt the body considerably. In addition, this type of remodeling is slow, tedious and expensive.

A subject of the invention is thus a composition, for example a cosmetic composition such as a make-up composition for keratin materials such as the skin and/or the lips and/or superficial body growths, which can allow these drawbacks to be overcome.

The inventor has found, surprisingly, that the combination of at least one organic polymer in a mixture of volatile and nonvolatile solvents allows a deposit to be placed on keratin materials, the volume of which increases over time without the intervention of an external stimulus, such as wetting the deposit, thus allowing the defects in the appearance of the keratin materials (e.g., blemishes, blackheads, dark shadows, folds, hollows and thinness) to be kept camouflaged for a long time.

A subject of the invention is thus a composition, such as a cosmetic composition, comprising at least one organic polymer, at least one volatile solvent which is incompatible with the organic polymer, and at least one nonvolatile solvent which is compatible with the organic polymer.

For the purposes of the invention, the expression “volatile solvent” is a nonaqueous medium which is liquid at room temperature (about 25° C.) and at atmospheric pressure (about 76 mmHg) and which is capable of evaporating completely from keratin materials. These solvents can be chosen, for example, from compounds having a nonzero vapor pressure, such as compounds having a vapor, pressure ranging from about 10⁻³mmHg to about 300 mmHg (at room temperature and atmospheric pressure).

For the purposes of the invention, the expression “nonvolatile solvent” is a nonaqueous medium which is liquid at room temperature and atmospheric pressure, for example, a liquid fatty substance (also referred to as an oil) which does not evaporate from keratin materials. These solvents can be chosen, for example, from substances having a vapor pressure of greater than about 300 mmHg (at room temperature and atmospheric pressure). According to the invention, it is possible to use one or more volatile solvents and one or more nonvolatile solvents.

According to the invention, the nonvolatile solvents are compatible with the polymer, i.e., capable of dissolving it at room temperature. The polymer can be an agent for gelling these nonvolatile solvents. Conversely, the volatile solvents in the composition of the invention are incompatible with the polymer, and are incapable of dissolving it: in other words, the polymer is not an agent for gelling the volatile solvents. The volatile and nonvolatile solvents of the invention are generally such that they allow the polymer to swell by dissolving it in the nonvolatile solvents gradually as the volatile solvents evaporate from the support on which the composition is deposited. Gradually as the volatile solvents evaporate off, the polymer passes from the insoluble state to the soluble state and deploys its fatty chains and forms a crosslinked or overlapping network trapping the nonvolatile solvents.

In U.S. Pat. No. 5,849,275, the disclosure of which is incorporated by reference herein, the polymers of the silicone/methacrylic type described are soluble in the volatile solvents indicated, and are thus mutually compatible. In addition, after these volatile solvents have evaporated off, the polymers precipitate in the nonvolatile oils of the paraffin or lanolin type, which corresponds to an insolubility of these polymers in these nonvolatile oils. It is this precipitation which gives the composition described transfer-resistance properties.

Moreover, in patent application EP0 850 644, the disclosure of which is incorporated by reference herein, it is indicated that the oils present in the composition should not be volatile and that they are trapped in the polyorganosiloxane. The indicated reason for this is that these oils should remain on the skin in order to treat it. Thus, the polyorganosiloxane is compatible with the oils or the nonvolatile solvents. It is also compatible with the volatile oils mentioned.

As volatile solvents which can be used in the invention, mention maybe made of water, volatile hydrocarbon-based oils, volatile silicone oils optionally comprising alkyl or alkoxy groups that are pendant or at the end of a silicone chain, and volatile fluoro oils, and mixtures thereof. Apolar volatile solvents can be used.

As volatile oils which can be used in the invention, mention may be made of linear or cyclic silicones comprising 2 to 7 silicon atoms, these silicones optionally comprising alkyl or alkoxy groups comprising 1 to 10 carbon atoms that are pendant or at the end of a chain, C₈ to C₁₆ isoparaffins, and C₅ to C₈ perfluorohydrocarbon-based oils. Representative volatile oils which may be mentioned include: octamethylcyclotetrasiloxane, decamethylcyclopentasioxane, dodecamethyl-cyclohexasiloxane, heptamethylhexyltrisiloxane, hepta-methylotyltnsiloxane, octamethyltrisiloxane and octamethyltetrasiloxane; C₈ to C₁₆ isoparaffins such as “Isopar”s and Permetyls, for example, isododecane; perfluoropolyethers comprising 5 to 8 carbon atoms, such as nonafluornmethoxybutane, nonafluoroethoxybutane, perfluoromethylcyclopentane and dodecafluoropentane; and mixtures thereof.

The amount of volatile solvent used depends on the desired magnitude of the volume-effect phenomenon. In practice, it is generally present in an amount ranging from about 0.1% to about 99.3% by weightrelative to the total weight of the composition, in some embodiments, from about 10% to about 80%, and in other embodiments, from about 20% to about 75%.

As nonvolatile solvents which can be used in the invention, mention may be made of polar oils such as:

hydrocarbon-based oils of animal origin, such as perhydrosqualene;

hydrocarbon-based plant oils such as liquid triglycerides of fatty acids and of glycerol, in which the fatty, acids may have varied chain lengths, these chains being linear or branched, and saturated or unsaturated; these oils can be chosen, for example, from wheatgerm oil, sunflower oil, corn oil, soybean oil, marrow oil, grapeseed oil, blackcurrant seed oil, sesame oil, hazelnut oil, apricot oil, macadamia oil, castor oil, avocado oil, karite butter, sweet almond oil, cotton oil, alfalfa oil, poppy oil, pumpkin oil, evening primrose oil, millet oil, barley oil, quinoa oil, olive oil, rye oil, safflower oil, candlenut oil, passion flower oil, musk rose oil and caprylic/capric acid triglycerides such as those sold by the company Stearineries Dubois or those sold under the names Miglyol 810, 812 and 818 by the company Dynamit Nobel;

natural or synthetic esters of formula R₁COOR₂, wherein R₁ is a higher fatty acid residue comprising 7 to 19 carbon atoms, and R₂ is a branched hydrocarbon-based chain comprising 3 to 20 carbon atoms, such as, for example, purcellin oil (cetostearyl octanoate), isopropyl myristate and alkyl or polyalkyl octanoates, decanoates or ricinoleates;

synthetic ethers of formula R³COR⁴, wherein R³ is a C₃ to C₁₉ alkyl radical, and R⁴ is a C₃ to C₂₀ alkyl radical;

fatty alcohols comprising at least 12 carbon atoms, such as octyldodecanol or oleyl alcohol;

cyclic hydrocarbons such as (alkyl)cycloalkanes, wherein the alkyl chain is linear or branched, saturated or unsaturated and comprises 1 to 30 carbon atoms, such as cyclohexane or dioctyl-cyclohexane;

aromatic hydrocarbons, for example, alkenes such as benzene, toluene, 2,4dimethyl-3-cyclohexene, dipentene, p-cymene, naphthalene or anthracene, and esters such as isosteaeryl benzoate;

primary, secondary or tertiary amines such as triethanolamine; and

mixtures thereof.

In one embodiment, synthetic esters such as isopropyl myristate are used.

The amount of these nonvolatile solvents depends on the amount of polymer used and on the magnitude of the desired volume effect. In practice, the nonvolatile solvent(s) are generally present in an amount ranging from about 0.2% to about 99.4% by weight relative to the total weight of the composition, in some embodiments, from about 5% to about 90%, and in other embodiments, from about 20% to about 85%.

It is possible to add one or more nonvolatile apolar oils to the above polar nonvolatile solvents, the apolar oils being chosen, for example, from: silicone oils such as polydimethylsiloxanes that are liquid at room temperature, phenyidimethicones, phenyltmethicnes, polymethylphenylsiloxanes, alkylpolydimethylsiloxanes comprising a C₂ to C₂₀ alkyl chain, and mixtures thereof; and linear or branched hydrocarbons of synthetic or mineral origin, for example, nonvolatile liquid paraffins and derivatives thereof, petroleum jelly, polydecenes, hydrogenated polyisobutene such as parleam, and mixtures thereof. These apolar oils can be used as the sole nonvolatile solvent or in a mixture of nonvolatile solvents.

In one embodiment, the apolar nonvolatile oils are dissolved by the polar nonvolatile solvents, with the whole generally representing from about 0% to about 80% by weight relative to the total weight of the composition, and in some embodiments, from about 30% to about 80%.

The nature of the nonvolatile solvents and that of the volatile solvents depend on the polymer used. For example, when the polymer is a crosslinked polydimethylsiloxane such as those described in patent application EP-A-0 850 644, the disclosure of which is incorporated by reference herein, for example those sold under the brand name KSG 6 or 16 by the company Shin Etsu or Trefil E-505C or 506C by the company Dow Corning, the apolar silicone oils mentioned above may be used as nonvolatile solvents, and volatile hydrocarbon-based oils such as C₈ to C₁₆ isoparaffins of the lsopar or Permetyl type, for example, isododecane, may be used as volatile solvent.

In other words, the nonvolatile solvents are good solvents for the polymer and the volatile solvents are poor solvents for the polymer.

The organic polymer can be a nonionic radical-mediated polymer belonging to the family of oil-superabsorbent materials, i.e., being able to take up as much as about 50 times its own weight of nonvolatile solvent. It is chosen, for example, from styrene homopolymers and copolymers and acrylic homopolymers and copolymers, and combinations thereof. In one embodiment, the polymer is a homqpolymer or copolymer of at least one monomer chosen from styrene, alkyl styrenes wherein the alkylgroup can be linear or branched and comprises 1 to 10 carbon atoms, alkyl (meth)acrylates wherein the alkyl group can be linear or branched and comprises 1 to 10 carbon atoms, and combinations thereof. The polymer can be chosen, for example, from styrene/alkyl (meth)acrylates, poly(alkylstyrene)s and alkylstyrene/alkyl acrylates. The alkyl (meth)acrylates can be chosen from 2-ethylhexyl acrylate and isobutyl methacrylate. As examples of polymers which can be used in the invention, mention may be made of methylstyrene/2-ethylhexyl acrylate and styrene/2-ethylhexyl acrylate/isobutyl methacrylate copolymers and poly(alkylstyrene)s.

The polymer can be in crosslinked form. Crosslinking agents which may be used, for example, are monomers comprising at least 2 optionally conjugated ethylenic bonds. By way of example, mention may be made of polyenes such as butadiene, bismaleimide or bisacrylamide. This crosslinking agent is generally present in an amount which allows maximum crosslinking of the polymer, for example in a content ranging from about 0.1% to about 5% by weight of the mixture (polymer/crosslinking agent).

Advantageously, the composition of the invention comprises at least one polymer which has a glass transition temperature (Tg) of less than 60° C., such as ranging from −100° C. to 60° C. The at least one polymer may be in the form of a dry powder having, for example, a particle size ranging from about 5 μm to about 600 μm, and in some embodiments from about 10 μm to about 450 μm.

As examples of crosslinked organic polymers which can be used in the invention, mention may be made of the commercial products Pliolite AC3-H (methylstyreney2-ethylhexyl acrylate, with a Tg of 58-64° C., at 100% active material) and AC5-G (styrene/2-ethylhexyl acrylate/isobutyl methacrylate, with a Tg of 49-550C at 100% active material) which are sold by the company Goodyear, and the products Imbiber Beads (mixture of kaolin and of poly(alkylstyrene) with a particle size of 125-420 μm, in a 1/99 weight ratio) and Polymer 295, (poly(alkylstyrene) with a particle size of 10-15 μm, at 100% solids) which are sold by the company Imbibitive Technologies.

The amount of polymers can be such that, after the volatile solvents have evaporated off and possibly some or all of the formula has penetrated into the keratin materials, the deposit remaining on these keratin materials comprises at least about 5% of the total weight of the polymer combined with the solvents and nonvolatile oils. In practice, these polymers can generally be present, in terms of active material in an amount ranging from about 0.5% to about 80% by weight relative to the total weight of the composition, and in some embodiments from about 5% to about 60%.

The invention can be used in make-up products for the skin of both the human face and body, including the scalp and the inner edge of the eyelids, the lips and superficial body growths such as the eyelashes, the eyebrows, the hair and the nails of human beings, and also in care and/or treatment products for the skin, the lips and superficial body growths.

The composition of the invention can be in the form of a paste, a solid or a cream. It can be a simple or multiple emulsion, for example, an oil-in-water or water-in-oil emulsion, or a solid or supple anhydrous gel. It can be in the form of an anhydrous gel. A gel has a consistency such that the composition does not flow under its own weight and such that the modulus of elasticity G* is greater than the viscous modulus G. This gel generally has a viscosity of greater than 1000 pascals, measured at 25° C. with a Haake rheometer.

The composition can also be in solid form. For example, it can be cast as a stick or a dish.

The invention can also be in the form of a treating or nontreating, coloring or noncolqring shampoo, or a hair conditioning product.

The composition according to the invention can be in the form of a dermatological or care composition for the skin, the lips or superficial body growths or in the form of an antisun composition or a make-up-removing composition. It can be in uncolored form, optionally comprising cosmetic or dermatological active agents. It can be used as a care base for the skin or the lips (e.g., a lip balm for protecting the lips against the cold and/or sunlight and/or the wind, or a day or night cream) or a care base for the nails.

The composition of the invention can also be in the form of a colored make-up product for the skin, for example a foundation, a concealer product, an eyeliner, a face powder, an eye shadow or a make-up product for the lips such as a lipstick or lip gloss, which optionally has care or treating properties, or alternatively a make-up product for keratin fibers, such as mascaras.

Needless to say, the composition of the invention is generally cosmetically or dermatologically acceptable, i.e., nontoxic and able to be applied to the skin, superficial body growths or the lips of human beings.

The composition of the invention can further comprise a dyestuff chosen from water-soluble and liposoluble dyes, pigments and nacres, and mixtures thereof. This dyestuff can be present in an amount ranging from about 0.01 to about 50% by weight relative to the total weight of the composition, and in some embodiments from about 1% to about 40%.

The composition can also comprise a particulate phase, which is generally present in a proportion ranging from about 0% to about 60% by weight relative to the total weight of the composition, and in some embodiments, from about 5% to about 35%. This particulate phase can comprise, besides pigments and/or nacres, fillers usually used in cosmetic or dermatological compositions.

The term “pigments” should be understood as meaning white or colored, mineral or organic particles, which are insoluble in liquid fatty phase and which are intended to color and/or opacify the composition. The term “fillers” should be understood as meaning colorless or white, mineral or synthetic, lamellar or nonlamellar particles. The term “nacres” should be understood as meaning iridescent particles, for example those produced by certain molluscs in their shell, or alternatively, those that are synthesized. These fillers and nacres can serve to modify the texture of the composition. For example, they can be structuring agents capable of giving a solid form.

The pigments maybe present in the composition in a proportion ranging from about 0.05% to about 50% by weight relative to the weight of the final composition, and in some embodiments, in a proportion ranging from about 2% to about 30%. As mineral pigments which can be used in the invention, mention may be made of titanium oxide, zirconium oxide and cerium oxide, as well as zinc oxide, ironoxide, chromium oxide and ferric blue. Among the organic pigments which can be used in the invention, mention may be made of carbon black and the lakes of barium, strontium, calcium (DC Red No. 7) and aluminum (DC Red No. 21 or FDC Yellow No. 6).

The nacres can be present in the composition in a proportion ranging from about 0% to about 30% by weight relative to the total weight of the composition, and in some embodiments, in a content ranging from about 1% to about 15%. Among the nacres which can be used in the invention, mention may be made of mica coated with titanium oxide, with iron oxide, with natural pigment and with bismuth oxychloride, such as colored titanium mica.

The fillers can be present in a proportion ranging from about 0% to about 60% by weight relative to the total weight of the composition, and in some embodiments from about 0.5% to about 20%. Mention may be made of talc, mica, kaolin, Nylon (for example Orgasol) powder, polyethylene powder, Teflon, starch, boron nitride, copolymer microspheres such as Expancel (Nobel Industrie), Polytrap (Dow Coring) and silicone resin microbeads (for example Tospeard from Toshiba).

The composition of the invention can also contain at least one wax, for example, to rigidify it. The wax(es) may be hydrocarbon-based waxes, fluoro waxes and/or silicone waxes and may be of plant, mineral, animal and/or synthetic origin. In one embodiment, the waxes have a melting point of greater than about 45° C.

As waxes which can be used in the invention, mention may be made of hydrocarbon-based waxes, silicone waxes and/or fluoro waxes optionally comprising ester, hydroxyl or thiol functions. Examples which may be mentioned are lanolin, beeswax, carnauba wax, candelilla wax, paraffin, lignite wax, microcrystalline wax, ceresin or ozokerite, synthetic waxes such as polyethylene waxes, silicone waxes such as alkyl- or alkoxydimethicone comprising 16 to 45 carbon atoms, Fischer-Tropsch waxes, and mixtures thereof.

The nature and amount of the waxes used depend on the desired mechanical and texture properties. As a guide, the composition can generally contain from about 0% to about 50% by weight of waxes, relative to the total weight of the composition, and in some embodiments from about 5% to about 30%. These waxes can also be agents for structuring the composition.

The composition of the invention can also comprise one or more additives commonly used in the field concerned, such as antioxidants, essential oils, preserving agents, neutralizing agents, cosmetic or dermatological active agents such as, for example, emollients, moisturizers, vitamins, essential fafty acids, sunscreens and aqueous-phase gelling agents. These additives can be present in the composition in the usual concentrations, for example, in a proportion ranging from about 0% to about 20% relative to the total weight of the composition, and in some embodiments from about 0% to about 10%.

Needless to say, a person skilled in the art will take care to select the optional additional additives and/or the amount thereof such that the advantageous properties of the composition according to the invention are not, or are not substantially, adversely affected by the addition envisaged.

The composition according to the invention can be manufactured by the known processes generally used in cosmetics or dermatology.

Another subject of the invention is a cosmetic process for caring for, making up or treating keratin materials such as the skin, the lips or superficial body growths such as the eyelashes and the hair human beings, this process comprising the application of the composition defined above, to the keratin materials.

Another subject of the invention is the use of at least one organic polymer combined with at least one volatile solvent which, is incompatible with the organic polymer and with a nonvolatile solvent which is compatible with the organic polymer, in a cosmetic composition or for the manufacture of a physiologically acceptable composition for topical application, to remodel the face and/or the body, for example the lips of the face, and/or to increase the volume of the lips of the face and/or to camouflage the esthetic imperfections and/or defects of keratin materials and/or to unify the complexion, for example in a long-lasting manner.

The invention is illustrated in greater detail in the non-limiting examples which follow. The percentages are given on a weight basis.

Example 1: Lip Composition

| | |
|---|---|
| Pliolite AC3-H | 9% |
| Isopropyl myristate | 81% |
| Isododecane | 10% |

Preparation: The polymer was added to the mixture of volatile and nonvolatile oils at room temperature, after heating it to 60-80° C. while, bubbling nitrogen through. A fluid mixture was obtained.

The volume of the polymer in the gel was 9% of the total volume. The volume of the polymer after evaporation of the isododecane was 100% of the total volume of the deposit. On applying this composition to the lips, they were observed to swell or to be made smooth after a few minutes, i.e., after evaporation of at least some of the isododecane. This effect was visible to the naked eye, by comparison with the application of a film-forming polymer alone which is soluble in isododecane, which gave a film that thins down in the course of the drying, such as, for example, long-chain alkanes or polymers that are dispersed and surface-stabilized by a stabilizer such as Kraton in isododecane (see patent application EP-A-0 749 747, the disclosure (of which, is incorporated by reference herein).

Example 2: Lip Composition

| | |
|---|---|
| Wax | 15% |
| Pigments | 9% |
| Pliolite AC3-H | 7% |
| Isopropyl myristate | 61% |
| Isododecane | 8% |

This lipstick was manufactured as in Example 1.

What is claimed is:

1. A composition comprising at least one organic polymer, at least one volatile solvent which is incompatible with said at least one organic polymer, and at least one nonvolatile solvent which is compatible with said at least one organic polymer.

2. A composition according to claim 1, wherein said at least one volatile solvent is an aqueous or nonaqueous medium with a vapor pressure ranging from 10⁻³ mmHg to 300 mmHg.

3. A composition according to claim 1, wherein, said at least one volatile solvent is chosen from water, volatile hydrocarbon-based oils, volatile silicone oils, and volatile fluoro oils.

4. A composition according to claim 3, wherein said volatile silicone oils comprise groups chosen from alkyl and alkoxy groups which can be pendant or at the end of the silicone chain.

5. A composition according to claim 1, wherein said at least one volatile solvent is an apolar solvent.

6. A composition according to claim 1, wherein said at least one volatile solvent is chosen from water, linear and cyclic silicones comprising 2 to 7 silicon atoms, C₈ to C₁₆ isoparaffins, and C₅ to C₈ perfluorohydrocarbon-based oils.

7. A composition according to claim 6, wherein said linear and cyclic silicones comprise alkyl or alkoxy groups comprising 1 to 10 carbon atoms which can be pendant or at the end of the silicone chain.

8. A composition according to claim 1, wherein said at least one volatile solvent is present in an amount ranging from 0.1% to 99.3% by weight relative to the total weight of the composition.

9. A composition according to claim 8, wherein said at least one volatile solvent is present in an amount ranging from 10% to 80% by weight relative to the total weight of the composition.

10. A composition according to claim 1, wherein said at least one nonvolatile solvent is polar.

11. A composition according to claim 1, wherein said at least one nonvolatile solvent is a nonaqueous medium which is liquid at room temperature.

12. A composition according to claim 1, wherein said at least one nonvolatile solvent is chosen from:
hydrocarbon-based oils of animal origin;
hydrocarbon-based plant oils;
natural and synthetic esters of formula R₁COOR₂, wherein R₁ is a higher fatty acid residue comprising 7 to 19 carbon atoms, and R₂ is a branched hydrocarbon-based chain comprising 3 to 20 carbon atoms;
synthetic ethers of formula R³COR⁴, wherein R³ is a C₃ to C₁₉ alkyl radical, and R⁴ is

16. A composition according to claim 12, wherein said natural and synthetic esters of formula R₁COOR₂ are chosen from purcellin oil (cetostearyl octanoate), isopropyl myristate, alkyl and polyalkyl octanoates, alkyl and polyalkyl decanoates, and alkyl and polyalkyl ricinoleates.

17. A composition according to claim 12, wherein said fatty alcohols comprising at least 12 carbon atoms are chosen from octyldodecanol and oleyl alcohol.

18. A composition according to claim 12, wherein said cyclic hydrocarbons are (alkyl)cycloalkanes, wherein the alkyl chain can be linear or branched, saturated or unsaturated, and comprises from 1 to 30 carbon atoms.

19. A composition according to claim 18, wherein said cyclic hydrocarbons are chosen from cyclohexane and dioctyl-cyclohexane.

20. A composition according to claim 12, wherein said aromatic hydrocarbons are chosen from aromatic alkenes and aromatic esters.

21. A composition according to claim 20, wherein said aromatic alkenes are chosen from benzene, toluene, 2,4-dimethyl-3-cyclohexene, dipentene, p-cymene, naphthalene and anthracene.

22. A composition according to claim 20, wherein said aromatic esters are isostearyl benzoate.

23. A composition according to claim 12, wherein said tertiary amines are triethanolamine.

24. A composition according to claim 12, wherein said silicone oils are chosen from polydimethylsiloxanes that are liquid at room temperature, phenyldimethicones, phenyltrimethicones, polymethylphenylsiloxanes, and alkylpolydimethylsiloxanes comprising a C₂ to C₂₀ alkyl chain.

25. A composition according to claim 1, wherein said at least one nonvolatile solvent is present in an amount ranging from 0.2% to 99.4% by weight relative to the total weight of the composition.

26. A composition according to claim 25, wherein said at least one nonvolatile solvent is present in an amount ranging from 5% to 90% by weight relative to the total weight of the composition.

27. A composition according to claim 1, wherein said at least one organic polymer is in crosslinked form.

28. A composition according to claim 1, wherein said at least one organic polymer is a nonionic radical-mediated polymer belonging to a family chosen from oil-superabsorbent materials and crosslinked polydimethyl-siloxanes.

29. A composition according to claim 1, wherein said at least one organic polymer is chosen from homopolymers and copolymers of at least one monomer chosen from styrene, alkylstyrenes wherein the linear or branched alkyl group comprises 1 to 10 carbon atoms, and alkyl (meth)acrylates wherein the linear or branched alkyl group comprises 1 to 10 carbon atoms.

30. A composition according to claim 1, wherein said at least one organic polymer is chosen from methylstyrene/2-ethylhexyl acrylate and styrene/2-ethylhexyl acrylate/isobutyl methacrylate copolyrmers and poly(alkylstyrene)s.

31. A composition according to claim 1, wherein said composition comprises at least one organic polymer which has a glass transition temperature (Tg) of less than 60° C.

32. A composition according to claim 1, wherein said at least one organic polymer, in terms of active material, is present in an amount ranging from 0.5% to 80% by weight relative to the total weight of the composition.

33. A composition according to claim 32, wherein said at least one organic polymer, in terms of active material, is present in an amount ranging from 5% to 60% by weight relative to the total weight of the composition.

34. A care and/or treating product for the skin, the lips and superficial body growths, or a make-up product for the skin, the lips or superficial body growths, comprising a composition which comprises at least one organic polymer, at least one volatile solvent which is incompatible with said at least one organic polymer, and at least one nonvolatile solvent which is compatible with said at least one organic polymer.

35. A composition according to claim 1, wherein said composition is in the form of an anhydrous gel.

36. A composition according to claim 1, wherein said composition is in solid form.

37. A composition according to claim 1, wherein said composition further comprises at least one additive chosen from dyestuffs, antioxidants, essential oils, preserving agents, neutralizing agents, cosmetic and dermatological active agents, aqueous-phase gelling agents, and fillers.

38. A composition according to claim 37, wherein said composition comprises at least one dyestuff.

39. A composition according to claim 1, wherein said composition further comprises at least one wax.

40. A composition according to claim 1, wherein said composition is effective to remodel the face and/or the body and/or to increase the volume of the lips of the face and/or to camouflage the aesthetic imperfections and/or defects of keratin materials and/or to unify the complexion.

41. A composition according to claim 40, wherein said composition is effective to remodel the lips of the face.

42. A composition according to claim 40, wherein said composition is effective to unify the complexion in a long-lasting manner.

43. A cosmetic process for caring for, making up or treating a keratin material, comprising applying a composition to said keratin-material, wherein said composition comprises at least one organic polymer, at least one volatile solvent which is incompatible with said at least one organic polymer, and at least one nonvolatile solvent which is compatible with said at least one organic polymer.
